(19) United States
(12) Patent Application Publication
(10) Pub. No.: US 2026/0060928 A1
(43) Pub. Date: Mar. 5, 2026

(54) TALC-FREE ACTIVE INGREDIENT COATING

(71) Applicant: Hermes Pharma GmbH, Pullach (DE)

(72) Inventors: Marieke GOSAU, Pullach (DE); Josef HAALA, Oberammergau (DE); Gerd WEISS, Munich (DE)

(21) Appl. No.: 19/107,040

(22) PCT Filed: Oct. 20, 2023
(86) PCT No.: PCT/EP2023/079345

§ 371 Date: Feb. 27, 2025

(30) Foreign Application Priority Data
Oct. 20, 2022 (EP) 22202757.5

(51) Int. Cl. A61K 9/16; A61K 31/137; A61K 31/4045; A61K 31/714; A61K 36/53; A61K 45/06

(52) U.S. Cl. CPC A61K 9/1623; A61K 9/1676; A61K 9/1682; A61K 31/137; A61K 31/4045; A61K 31/714; A61K 36/53; A61K 45/06

(57) ABSTRACT

The invention provides a dry, flowable composition in the form of a plurality of particles, each particle comprising, or consisting of, an inert starter core which is coated with at least one coat of an active ingredient composition. The particles are characterized in that the active ingredient composition is substantially free of anti-tacking agents such as talc, and the inert starter core comprises, or consists of, a sugar alcohol wherein the sugar alcohol is mannitol, in particular, a mannitol provided in a direct compression quality. The invention further provides a process for the preparation of the dry, flowable composition, and more specifically a process of coating inert starter cores with an active ingredient coat without the use of anti-tacking agents, such as talc.

### BACKGROUND OF THE INVENTION

The present invention relates to dry, flowable compositions provided in the form of particles, or formulations based thereon, wherein the particles comprise an active ingredient coated onto inert starter cores; typically starter cores with a particle size of below 1000 μm. Oftentimes, further coats of materials such as polymers, lipids and/or waxy substances, which are modulating the release of the active ingredient and/or protecting it from external conditions, are subsequently applied on top of this active ingredient coat. This type of composition is sometimes also referred to as a multi-particulate system, or—when working with spherical or spheroidal starter cores (pellets)—also as a multiple unit pellet system (‘MUPS’).

Fluidized bed coaters are one of the most common devices used to apply the active ingredient to inert starter cores, for instance, in the form of a solution or suspension of the active ingredient in an aqueous, hydro-alcoholic or organic diluent. Commonly, a binder is employed in order to improve adhesion of the active ingredient to the inert core material, and thus improve coating efficiency. However, even though the fluidized bed approach reduces particle-to-particle contact by suspending them in an airstream, the coating process still requires a fine balance between air volume, product bed temperature, spray pressure and flow rate of the liquid active ingredient composition sprayed onto the fluidized particles, to prevent, or lower the risk of, particles agglomerating at their wetted, sticky surfaces. Typically, anti-tacking agents (also, anti-tack agents) such as talc, magnesium stearate, or the like, are added to the liquid active ingredient composition to lower the risk of particle agglomeration further; especially, in industrial settings where large amounts of the liquid active ingredient composition need to be sprayed efficiently in short time periods.

One of the most commonly used anti-tacking agents active-ingredient coating processes is talc, or talcum. Oftentimes between 5 to 150 wt.-% talc are employed, sometimes up to 200 wt.-%, based on the weight of the active ingredient to be coated onto a core material. While common, the use of talc can be challenging, though, especially in production scale, due to sedimentation issues; for instance, talc sedimenting in the tubes or pipelines transporting liquid active ingredient compositions to an outlet such as a spray nozzle. Moreover, talc can lead to frequent nozzle clogging in the spraying process, a problem often aggravated by the afore-mentioned sedimentation.

In addition, recent publications such as WO 2022/078823 A1 (WO'823) also describe that there are growing safety concerns associated with the use of talc-containing coatings systems in the dietary supplement/nutraceutical industries, and highlight the need to develop talc-free coating systems. Yet, unlike the present invention, WO'823 addresses this object by replacing talc with another anti-tacking agent, namely ground-up outer coats of grain seeds, such as rice husk. The future has to show if this approach is transferrable to active ingredient coatings directly onto an inert starter core (as opposed to the outer top-coating applied over an otherwise prepared active ingredient containing core as in WO'823).

U.S. Pat. No. 7,105,180 B2 (US'180) also notes that talc may contain impurities, and can cause a pH of more than 7 in suspensions of talc in water. While it remains unclear from US'180, if this is merely a general observation on talc or might in fact be seen as detrimental to the stability of some active ingredients, the authors of US'180 proceed to describe a lab-scale dispersion coating process to apply omeprazole to neutral pellets which appears to be free of talc. However, no information is made available regarding the nature of these neutral pellets, nor the processing conditions. In other words, it remains unclear, for instance, how efficient this lab-scale process was in terms of time or unwanted agglomeration, if this process would be transferrable to industrial scale, and, if so, which parameter or material selection rendered this process of US'180 possible without the use of talc.

US 2010/0080849 A1 (US'849) also describes an example of a talc-free, lab-scale active ingredient coating process for duloxetine, and employs specific mannitol pellets as the starter core material, the optimized preparation of which in a fluidized-bed coater by continuously spraying and drying a mannitol solution or dispersion is the main focus of US'849. Nothing in US'849 suggests, however, a link between the use of mannitol and the capability to omit the use of talc in active ingredient coating processes. Instead US'849 is mainly concerned with optimizing sphericity, hardness, and surface smoothness of their mannitol pellets and their general suitability as starter cores.

Moreover, US'849 employs a 1:1 water/ethanol vehicle when applying their duloxetine/binder solution. Using solvents other than water, for instance alcohols such as ethanol, can inherently reduce the surface-stickiness of the coated particles without the use of talc but comes at the price of increased occupational hazards and reduced cost-efficiency (purchase and recovery requirements of the alcohol).

It is thus an object of the present invention to provide active-ingredient coated particles, based on inert starter cores, that are substantially free of anti-tacking agents, such as talc, and processes for their preparation. Further objects of the invention will be clear on the basis of the following description of the invention, examples and claims.

### SUMMARY OF THE INVENTION

In a first aspect, the invention relates to a dry, flowable composition provided in the form of a plurality of particles, wherein the particles each comprise, or consist of, an inert starter core coated with at least one coat of an active ingredient composition, characterized in that the active ingredient composition is substantially free of anti-tacking agents, such as talc, and the inert starter core comprises, or consists of, a sugar alcohol, wherein the sugar alcohol is mannitol provided in a direct compression quality.

In a second aspect, the present invention provides a process for the preparation of a dry, flowable composition provided in the form of a plurality particles, wherein the particles each comprise, or consist of, an inert starter core coated with at least one coat of an active ingredient composition, the process comprising the steps of:
(a) Providing inert starter cores,
(b) Providing a liquid active ingredient composition,
(c) Spraying the liquid active ingredient composition onto the inert starter cores, and
(d) Drying the active ingredient coated particles obtained in step (c),
characterized in that the active ingredient composition is substantially free of anti-tacking agents, and the inert starter core comprises, or consists of, a sugar alcohol, wherein the sugar alcohol is mannitol provided in a direct compression quality.

### DETAILED DESCRIPTION OF THE INVENTION

In a first aspect, the invention relates to a dry, flowable composition provided in the form of a plurality of particles, wherein the particles each comprise, or consist of, an inert starter core coated with at least one coat of an active ingredient composition, characterized in that the active ingredient composition is substantially free of anti-tacking agents, and the inert starter core comprises, or consists of, a sugar alcohol, wherein the sugar alcohol is mannitol provided in a direct compression quality.

In other words, the invention relates to a dry, flowable composition of active ingredient containing particles based on inert sugar alcohol starter cores, namely mannitol starter cores, wherein the active ingredient coat applied onto said inert cores is substantially free of anti-tacking agents.

As used herein, the term ‘inert starter core’ shall be understood as a pharmaceutically acceptable and typically commercially available raw material in dry, flowable particulate form which serves as an inert carrier for the active ingredient coated thereon. The expression is not limited to a specific shape of the inert starter core, e.g., the invention does not require the inert starter cores to be spherical or spheroidal, or to exhibit smooth surfaces as commonly implied in the prior art, such as US'849, for multi-partiuclate systems. However, aggregation, or granulation, of the inert starter cores with the applied active ingredient composition is expressly not the aim of the present invention and should be avoided. Instead, the invention relates to particles with an active ingredient coat that is distinct from the inert starter core material underneath, not inter-mixed therewith.

Further, as used herein, the term ‘substantially free of X’ means that the respective material (e.g., a chemical compound or a composition) contains either no ‘X’ at all, or at least far less than a functional amount thereof, typically less than 1 wt.-%, preferably less than 0.1 wt.-%, or even less than 0.01 wt.-%, of the respective ingredient ‘X’. The expression refers, inter alia, to very small amounts of ‘X’, such as material inherent impurities or residual trace amounts of reactants, or moisture, which may potentially be present in (raw) materials despite the aim to render a material completely free of them. For example, ‘substantially free of water’ means that no water is deliberately included in a material but does not exclude the presence of residual moisture.

Similarly, the expression ‘the active ingredient is not A’, shall be understood to cover active ingredient A as such, as well as related compounds, such as pharmaceutically acceptable salts or hydrate forms thereof. In that regard, it shall further be understood that terms like ‘active ingredient’ (or sometimes ‘active pharmaceutical ingredient’ (API)), ‘active agent’, ‘bioactive agent’, ‘active principle’, or ‘drug’ are used synonymously and refer to a compound, or combination of compounds, which are active—either therapeutically or as a preventive measure—against an undesired condition in a subject, such as a human.

As used herein, all percentages, parts and/or ratios in the context of numbers should be understood as relative to the total number of the respective items, unless otherwise specified, or indicated or required by the context. Furthermore, all percentages, parts and/or ratios are intended to be by weight of the total weight; i.e., ‘%’ should be read as ‘wt.-%’, unless otherwise specified, or indicated or required by the context.

Further, unless specified otherwise, any measured provisions such as densities, solubulities, or the like, are understood to be measured at room temperature (i.e., 20±5° C. as defined by the European Pharmacopoeia or by the WHO guidance ‘Guidelines for the Storage of Essential Medicines and Other Health Commodities’ (2003)).

As used herein, the term ‘anti-tacking agent’ refers to substances that reduce stickiness, in particular surface stickiness, of materials in coating processes and thus prevent ‘tacking’ or ‘caking’ as well as excessive agglomeration. Examples of anti-tacking agents include talc, magnesium stearate, magnesium silicate, calicum silicate, colloidal SiO₂, and silica.

In a specific embodiment, the anti-tacking agent is talc; or in other words, the particles according to the first aspect of the invention are characterized in that their active ingredient coat is substantially free of talc.

According to the invention, the sugar alcohol is mannitol, and more specifically mannitol in a direct compression quality; or in other words, a quality, or grade, of the raw material that is explicitly offered by suppliers to be suited, in particular, for direct compression purposes or as a direct compression excipient.

Optionally, the inert starter core essentially consists of said mannitol. As used herein, the terms ‘consisting of’ or ‘essentially consisting of’ mean that no further components are added to a composition or dosage form other than those listed. This does not exclude the potential presence of very small amounts of other materials, such as material inherent impurities and/or processing aids not exceeding 1 wt-%, preferably not exceeding 0.5 wt-%, of the material in question. Furthermore, when referring herein to e.g., ‘essentially consisting of A, B, C, and optionally D’ this means that no further components are added to a composition or dosage form other than A, B, C and D, but with D still being an optional component (i.e., not mandatory) in said composition or dosage form.

Oftentimes, such direct compression raw material grades, such as mannitol in a direct compression quality, are characterized by an irregular shape with uneven, jagged, and often porous surfaces. These properties which enable the particles to interlock more efficiently with one another upon compression (thereby yielding sufficiently solid compacts at lower compression forces) also differentiate mannitol in a direct compression quality from, for instance, spheronized, or otherwise rounded, and more even-surfaced mannitol pellets, such as those described in US'849.

Examplary grades of commercially available mannitol in direct compression quality include Pearlitol® 200 SD, Pearlitol® 200 GT, or Pearlitol® 300 DC by Roquette, Parteck® M200 by Merck, as well as Mannogem® XL or Mannogem® XL Ruby by SPI Pharma. And while, for instance, US'849 favours sphericity values in terms of the length-width ratio of the particles to be less than about 1.05, Pearlitol® 300 DC—one of the preferred mannitol grades in direct compression quality—has a length-width ratio of 1.35±0.02 (n=6 batches; see example 5A). Mannogem® XL Ruby also exhibits a length-width ratio of 1.41±0.01 (n=3; see example 5B). Similarly, while US'849 favours a D10/D90 ratio of their particles to be 0.6-1.0, Pearlitol® 300 DC has a D10/D90 ratio of only 0.39±0.10 (n=6); Mannogem® XL Ruby of only 0.26±0.02 (n=3).

The inventors found that sugar alcohols such as mannitol, and in particular their respective raw materials in direct compression quality are suited as simple, cost-efficient inert starter core materials for active ingredient coating processes, especially active ingredient coating processes in conventional fluidized-bed settings. This is surprising insofar as particles exhibiting the irregular shape and rough surfaces typically associated with direct compression qualities are not commonly recommended for this purpose; likely because a more even, smooth, and reproducible active ingredient coat seems expectable in fluidized bed processes when using the more regularly shaped, and often spheronized, or otherwise rounded, starter cores that are commercially available and advertised for the exact purpose of active ingredient coating; for instance, spherical or spheroidal pellets based on sucrose, or microcrystalline cellulose, (e.g., Suglets®, Cellets®, or Celphere®). Even in most prior art documents, such as US'849, the authors aim to optimize sphericity, hardness, and surface smoothness of pellets that are meant to serve as starter cores for active ingredient coatings, with US'849 even employing specifically adapted fluidized-bed settings to achieve said features. In contrast, direct compression quality mannitol grades, such as the commercial ones mentioned above, are typically advertised for the preparation of tablet or lozenge formulations, and have no interest in providing overly spherical or smooth particles as this would negatively impact their direct compression properties.

Even more surprisingly, the inventors found that sugar alcohols, and in particular mannitol, serve(s) as a beneficial inert starter core material insofar as it can obviate the need for talc as anti-tacking agent in the active ingredient coating process. For instance, as shown in the examples below, when applying active ingredients coats of vitamin B12 or phenylephrine hydrochloride onto inert starter cores, only mannitol starter cores allowed the omittance of anti-tacking agents such as talc.

In one embodiment, the inert starter cores, or in other words a plurality of the inert starter cores, exhibit(s) a poured density (also referred to as poured bulk density, or freely settled bulk density, and occasionally just as bulk density) in the range of 0.40-0.80 g/mL, preferably from 0.55-0.80 g/mL, and further preferably from 0.60-0.80 g/mL. For instance, the starter cores may exhibit a poured density in the range of 0.40-0.55 g/mL such as 0.45 g/mL or 0.48 g/mL, or 0.52 g/mL, in the range of 0.55-0.65 g/mL, such as 0.56 g/mL, or in the range of 0.60-0.75 g/mL, such as 0.62 g/mL, 0.63 g/mL, 0.65 g/mL, or 0.69 g/mL.

Starter cores essentially consisting of directly compressible mannitol with a median particle size (D50), as determined by dynamic image analysis, of about 120-170 μm, and being prepared by spray-drying (e.g., Pearlitol® 200 SD) commonly exhibit a poured density of about 0.45-0.48 g/mL, occasionally about 0.45-0.52 g/mL; whereas mannitol starter cores with a median particle size (D50) in the same 120-170 μm-range but prepared by a granulation process (e.g., Pearlitol® 200 GT) commonly exhibit a higher poured density in the range of about 0.62-0.69 g/mL, such as 0.63 g/mL. Mannitol starter cores also prepared by a granulation process but exhibiting a slightly larger median particle size (D50) of about 200-350 μm (e.g., Mannogem® XL Ruby) commonly exhibit a poured density of about 0.55-0.60 g/mL; and those with a median particle size (D50) of about 280-350 μm and prepared by extrusion (e.g., Pearlitol® 300 DC) commonly exhibit a poured density of about 0.62-0.69 g/mL.

Alternatively, or in addition thereto, the inert starter cores, or in other words a plurality of the inert starter cores, exhibit(s) a tapped density (also referred to as tapped bulk density) in the range of 0.50-1.00 g/mL, preferably 0.60-1.00 g/mL, and further preferably from 0.65-1.00 g/mL. For instance, the starter cores may exhibit a tapped density in the range of 0.50-0.60 g/mL such as 0.52 g/mL or 0.57 g/mL, in the range of 0.60-0.70 g/mL, such as 0.63 g/mL or 0.66 g/mL, or in the range of 0.65-0.85 g/mL, such as 0.69 g/mL, 0.75 g/mL, or 0.79 g/mL.

Starter cores essentially consisting of directly compressible mannitol with a median particle size (D50), as determined by dynamic image analysis, of about 120-170 μm, and being prepared by spray-drying (e.g., Pearlitol® 200 SD) commonly exhibit a tapped density of about 0.52-0.57 g/mL, occasionally about 0.52-0.66 g/mL; whereas mannitol starter cores with a median particle size (D50) in the same 120-170 μm-range but prepared by a granulation process (e.g., Pearlitol® 200 GT) commonly exhibit a higher tapped density in the range of about 0.69-0.79 g/mL, such as 0.75 g/mL. Mannitol starter cores also prepared by a granulation process but exhibiting a slightly larger median particle size (D50) of about 200-350 μm (e.g., Mannogem® XL Ruby) commonly exhibit a tapped density of about 0.60-0.67 g/mL; and those with a median particle size (D50) of about 280-350 μm, and prepared by extrusion (e.g., Pearlitol® 300 DC) a tapped density of about 0.69-0.79 g/mL.

In one of the preferred embodiments, the inert starter core, or a plurality thereof, is/are prepared by an extrusion process, or involving an extrusion process. In a further preferred embodiment, the inert starter core, or a plurality thereof, is/are prepared by a granulation process, or involving a granulation process.

Further preferred is when the inert starter core(s) is/are not prepared by a powder coating process, and also not by a spray-drying process, or at least not solely by a spray-drying process. The authors found that extruded or granulated inert starter cores comprising, or consisting of sugar alcohols such as mannitol, were beneficial over e.g., spray-dried qualities insofar as the extruded or granulated qualities offer, inter alia, a higher mechanical stability. One example of a commercially available, extruded mannitol grade is the above-mentioned Pearlitol® 300 DC by Roquette. This material, which is advertised as a direction compression excipient for use in lozenges, swallowable tablets, orally dispersible tablets, chewable tablets, and effervescent tablets, yielded particularly good outcomes in terms of serving as an inert starter core and allowing for active ingredient coating without the need for anti-tacking agents such as talc. Moreover, the extruded grade performed even better than e.g., spray-dried mannitol grades such as Pearlitol® 200 SD or Parteck® M200 in these aspects.

Similar observations were made for the granulated mannitol grades in direct compression quality, such as Pearlitol® 200 GT by Roquette, or Mannogem® XL Ruby by SPI Pharma.

In one of the further preferred embodiments, the mannitol in the inert starter cores is present predominantly as its β-polymorph, e.g., with only trace amounts of 1% or lower of the α-polymorph or the δ-polymorph. This includes e.g., Pearlitol® 200 GT and Pearlitol® 300 DC whereas the spray-dried Pearlitol® 200 SD contains both mannitol's α and β-polymorphs.

In one embodiment, the inert starter core is substantially free of talc.

In one embodiment, a plurality of the inert starter cores exhibits a median particle size (D50) in the range of about 50-800 μm, or in the range of about 100-500 μm, or in the range of about 150-350 μm, or in the range of about 200-350 μm, as determined by dynamic image analysis, e.g., using using a Camsizer® XT device (Retsch Technology GmbH, Haan, Germany).

In one embodiment, the at least one coat of the active ingredient composition is applied directly onto the inert starter core, i.e., without any intermediate coats. For instance, no seal coats or other forms of stabilizing coats are applied prior to coating the inert starter cores with the active ingredient composition.

In one embodiment, the composition according to the first aspect of the invention is a pharmaceutical or nutraceutical composition.

In one embodiment, when measured in water at room temperature (i.e., 20±5° C.), the active ingredient is considered practically insoluble, very slightly soluble, slightly soluble, sparingly soluble, soluble, or freely soluble as per European Pharmacopoeia definitions (Ph.Eur. 10^{th} ed.; see below); preferably, practically insoluble, very slightly soluble, slightly soluble, sparingly soluble, or soluble.

| | Approximate volume of | |
|---|---|---|
| | solvent per 1 g of solute | |
| Very soluble | <1 | mL |
| Freely soluble | 1-10 | mL |
| Soluble | 10-30 | mL |
| Sparingly soluble | 30-100 | mL |
| Slightly soluble | 100-1000 | mL |
| Very slightly soluble | 1000-10000 | mL |
| Practically insoluble | >10000 | mL |

In one embodiment, when measured in water at room temperature, the active ingredient exhibits an aqueous solubility in the range of 0.01-1000 mg/ml; preferably 0.01-100 mg/mL.

In one embodiment, the active ingredient is selected from the group consisting of vitamins, plant extracts, sympathomimetics, analgesics, cough suppressants, antidiarrhoeals, sleep-inducing agents, antihistaminics, and proton-pump inhibitors. In a specific embodiment, the active ingredient is selected from the group consisting of vitamin B12, thyme dry extract, ivy dry extract, curcuma powder, phenylephrine hydrochloride, dextromethorphan hydrobromide, loratadin, cetirizine, omeprazole, and melatonin. In a more specific embodiment, the active ingredient is selected from the group consisting of vitamin B12 and phenylephrine hydrochloride.

In one embodiment, the active ingredient is not irbesartan, clopidogrel, diclofenac, duloxetine, or glimepiride; or in other words, the active ingredient is not an active ingredient selected from the group consisting of irbesartan, clopidogrel, diclofenac, duloxetine, and glimepiride. As mentioned above, this includes both the active ingredients as such, as well as their related compounds, such as pharmaceutically acceptable salts or hydrate forms of these active ingredients.

In one embodiment, the particles are prepared by a liquid coating process. This means that the at least one active ingredient coat on the inert starter cores is applied using a liquid coating composition which is gradually dried to solidity on the starter cores throughout the coating process, thereby gradually forming an active ingredient coat. In other words, the active ingredient coat is not applied, for instance, as a dry powder coating. Moreover, the particles being prepared by a liquid coating process in this embodiment, does not mean, or require, that the starter cores are also to be prepared by a liquid coating process, as suggested e.g., in US'849. As indicated above, starter cores with a high degree of sphericity and surface smoothness would not commonly qualify as direct compression quality materials.

In a specific embodiment, the active ingredient composition is provided in the form of a suspension or a solution of the active ingredient in a liquid diluent. In a more specific embodiment, the liquid diluent is water, or in other words, the liquid coating process is performed using a water-based coating composition of the active ingredient. In a yet more specific embodiment, the liquid diluent is substantially free of organic solvents; for instance, organic solvents such as ethanol, methanol, acetone, acetonitrile, chloroform, methylene chloride, ethylene glycol, dichloromethane, toluol, or the like. In a further embodiment, the particles are substantially free of organic solvents or residues thereof.

Purely water-based coating processes (i.e., those using no solvent other than water) are preferred insofar as they obviate certain occupational hazards, such as flammability, and/or the need for solvent-purchase and recovery typically involved with common non-aqueous solvents like alcohols (e.g., ethanol, isopropanol), or ketones (e.g., acetone). Unfortunately, purely water-based coating processes are also harder to manage, especially in industrial scale batches, when aiming to avoid the use of talc, since water—as a potent solvent for most, if not all, commercially employed starter core materials—tends to increase the surface stickiness of the starter cores, and thus agglomeration tendencies. Oftentimes, these agglomeration tendencies are then dealt with either talc and/or adding organic solvents such as those listed above. The inventors now surprisingly found that another valid approach to allow for talc-free active ingredient coating, even for purely water-based coating systems, resides in the choice of sugar alcohols, and in particular mannitol, in direct compression qualities (e.g., the commercial grades named exemplarily above) as the inert starter core material. This was found for systems both with or without a polymeric binder.

In one embodiment, the active ingredient composition is applied to the inert starter cores in a fluidized bed coater, e.g., in a conventional fluidized-bed coater such as a Ventilus® 2.5 bottom-spray fluidized bed coater, or the larger Ventilus® V 100 or V 400 bottom-spray fluidized bed coaters for industry-scale batches, such as about ≥50 kg per batch (all by Romaco Innojet, Germany).

In one embodiment, the active ingredient composition comprises an excipient selected from the group of binders, anti-foaming agents, stabilizers, buffers, emulsifiers, solubilizers, p, and anti-flocculation agents. For instance, water-soluble polymeric binders such as hydroxypropyl methylcellulose (HPMC) or polyvinylpyrrolidone (PVP), or anti-foaming agents such as simethicone or dimethicone may be used, as needed, to further improve the handling of the active ingredient composition, both during its preparation and application to the starter cores. In this regard, it shall be understood that while substances such as HPMC or simethicone are also known to have active ingredient properties on their own if administered in the right amount, within the framework of this invention, they are meant to be excipients only and are only employed in amounts that aid preparation and application of the liquid active ingredient composition to the starter cores, but that are too low to cause relevant physiological effects.

In one embodiment, the active ingredient content of the particles does not exceed 50 wt.-% based on the weight of the active ingredient coated particle.

In one embodiment, the active ingredient coated particles comprise at least one further coat of a coating composition, optionally a coat comprising, or consisting of, one or more polymers, waxes and/or lipids applied from aqueous, organic or hot-melt coating compositions. In a specific embodiment, the coated composition exhibits modified release properties, such as taste-masking, enteric release properties and/or sustained release properties.

In one embodiment, a single dose unit of the plurality of particles is provided in the form of a tablet compressed therefrom; or a hard-gelatine capsule, stick-pack, sachet, or vial filled with them.

In a second aspect, the present invention provides a process for the preparation of a dry, flowable composition provided in the form of a plurality particles, wherein the particles each comprise, or consist of, an inert starter core coated with at least one coat of an active ingredient composition, the process comprising the steps of:
(a) Providing inert starter cores,
(b) Providing a liquid active ingredient composition,
(c) Spraying the liquid active ingredient composition onto the inert starter cores, and
(d) Drying the active ingredient coated particles obtained in step (c),
characterized in that the active ingredient composition is substantially free of anti-tacking agents, and the inert starter core comprises, or consists of, a sugar alcohol, wherein the sugar alcohol is mannitol provided in a direct compression quality.

In other words, the present invention provides a process for the preparation of the dry, flowable composition in the form of a plurality particles according to the first aspect of the invention. Accordingly, any embodiments, including specific or preferred embodiments, disclosed herein-above in connection with the particles or the dry, flowable composition according to the first aspect of the invention, may be equally applied to the preparation process according to this second aspect of the invention; for instance, that in specific embodiments, the anti-tacking agent is talc; the inert starter core comprises, or consists of, mannitol in direct compression quality such as the commercially available grades Pearlitol® 200 SD, 200 GT or 300 DC grades by Roquette, or Mannogem® XL Ruby by SPI Pharma; the inert starter cores exhibit a poured density in the range of 0.40-0.80 g/mL, and/or a tapped density in the range of 0.50-1.00 g/mL, a median particle size (D50) in the range of about 50-800 μm, and are preferably prepared by an extrusion process or a granulation process (or by processes involving extrusion or granulation); or the composition is a pharmaceutical or nutraceutical composition. The provisions described above with respect to the active ingredient(s) applied to the inert starter core (e.g., nature or solubilities thereof) or the active ingredient composition (e.g., physical form, choice of diluents and/or excipients) also apply equally to the preparation process of the second aspect of the invention.

In one embodiment of the preparation process, the at least one coat of an active ingredient composition is applied directly onto the inert starter cores, i.e., without any intermediate coats.

In one embodiment of the preparation process according to the second aspect of the invention, the active ingredient composition is sprayed onto the inert starter cores in step (c) using a fluidized bed coater, e.g., in a conventional fluidized-bed coater such as a Ventilus® 2.5 bottom-spray fluidized bed coater, or the larger Ventilus® V 100 or V 400 bottom-spray fluidized bed coaters for industry-scale batches, such as about ≥50 kg per batch (all by Romaco Innojet, Germany. Fluidized-bed coaters are beneficial insofar as the large ‘air bed’ generated by the device allows for steps (c) and (d) to occur simultaneously, i.e., the starter cores are sprayed with the liquid active ingredient composition while being dried.

In a specific embodiment, the fluidized bed coater is a bottom-spray coater; for instance, for lab-scale batches a Ventilus® 2.5 bottom-spray fluidized bed coater, or for industry-scale batches such as about ≥50 kg per batch a Ventilus® V 100 or V 400 bottom-spray fluidized bed coater (both Romaco Innojet, Germany). The latter is preferred insofar as the inventors found the bottom-spray fluidized bed coaters to yield more uniform coatings than top-spray devices (mostly for the active ingredient coat but also—where present—for further coats applied atop of the active ingredient coat, e.g., coats with modified release properties) and less aggregation, or granulation. Aggregation, or granulation, of the inert starter cores with the applied active ingredient composition is not the aim of the present invention; instead, the investion relates to the preparation of particles with an active ingredient coat that is distinct from the inert starter core material underneath, not inter-mixed therewith.

In one embodiment, the spraying pressure in step (c) is in the range of from 1.0-3.0 bar, preferably 1.4-2.0 bar, such as 1.6 bar, 1.7 bar or 1.8 bar.

In one embodiment, when using mannitol cores in direct compression quality as the inert starter cores as described herein, the active ingredient coating can be applied by spraying at a maximum spray-rate in g/min per core weight which is at least 1.2× higher, or at least 1.5× higher, or at least 2.0× higher, compared to the same active ingredient coating using the same amount of sucrose-based starter cores instead of the mannitol ones.

In one embodiment, the active ingredient content of the particles does not exceed 50 wt.-% based on the weight of the active ingredient coated particle.

In an optional embodiment, the active ingredient coated particles obtained by the preparation process described above are top-coated with at least one further coating composition, for instance, a coating composition comprising, or consisting of, one or more polymers, waxes and/or lipids applied from aqueous, organic or hot-melt coating compositions. In a specific embodiment, the coated composition exhibits modified release properties, such as taste-masking, enteric release properties and/or sustained release properties.

In one embodiment, the active ingredient coated particles obtained by the preparation process described above are either compressed into tablets, or they are filled into a hard-gelatine capsule, stick-pack, sachet, or vial, so as to form a single dose unit of the composition according to the first aspect of the invention.

The following list of numbered items are embodiments comprised by the present invention:
1. A dry, flowable composition provided in the form of a plurality of particles, wherein the particles each comprise, or consist of, an inert starter core coated with at least one coat of an active ingredient composition, characterized in that the active ingredient composition is substantially free of anti-tacking agents, and the inert starter core comprises, or consists of, a sugar alcohol, wherein the sugar alcohol is mannitol provided in a direct compression quality.
2. The composition according to item 1, wherein the anti-tacking agent is talc.
3. The composition according to item 1 or 2, wherein the inert starter core essentially consists of the mannitol.
4. The composition according to any one of items 1 to 3, wherein a plurality of the inert starter cores exhibits a poured density in the range of 0.40 to 0.80 g/mL, preferably from 0.55 to 0.80 g/mL, and further preferably from 0.60 to 0.80 g/mL.
5. The composition according to any one of items 1 to 4, wherein a plurality of the inert starter cores exhibits a tapped density in the range of 0.50 to 1.00 g/mL, preferably from 0.60 to 1.00 g/mL, and further preferably from 0.65 to 1.00 g/mL.
6. The composition according to any one of items 1 to 5, wherein the inert starter core is prepared by an extrusion process, or involving an extrusion process.
7. The composition according to any one of items 1 to 6, wherein the inert starter core is prepared by a granulation process, or involving a granulation process.
8. The composition according to any one of items 1 to 7, wherein the inert starter core is not prepared by a powder coating process.
9. The composition according to any one of items 1 to 8, wherein the inert starter core is not prepared by a spray-drying process, or not solely by a spray-drying process.
10. The composition according to any one of items 1 to 9, wherein the inert starter core is substantially free of talc.
11. The composition according to any one of items 1 to 10, wherein a plurality of the inert starter cores exhibits a median particle size (D50) in the range of about 50-800 μm, or in the range of about 100-500 μm, or in the range of about 150-350 μm, as determined by dynamic image analysis, e.g., using using a Camsizer® XT device (Retsch Technology GmbH, Haan, Germany).
12. The composition according to any one of items 1 to 11, wherein the at least one coat of an active ingredient composition is applied directly onto the inert starter core, i.e., without any intermediate coats.
13. The composition according to any one of items 1 to 12, wherein the composition is a pharmaceutical or nutraceutical composition.
14. The composition according to any one of items 1 to 13, wherein, when measured in water at room temperature (20±5° C.), the active ingredient is considered practically insoluble, very slightly soluble, slightly soluble, sparingly soluble, soluble, or freely soluble as per Ph.Eur. ed.10 definitions; preferably, practically insoluble, very slightly soluble, slightly soluble, sparingly soluble, or soluble.
15. The composition according to any one of items 1 to 14, wherein, when measured in water at room temperature (20±5° C.), the active ingredient exhibits an aqueous solubility in the range of 0.01-1000 mg/ml; preferably 0.01-100 mg/mL.
16. The composition according to any one of items 1 to 15, wherein the active agent is selected from the group consisting of vitamins, plant extracts, sympathomimetics, analgesics, cough suppressants, antidiarrhoeals, sleep-inducing agents, antihistaminics, and proton-pump inhibitors.
17. The composition according to any one of items 1 to 16, wherein the active agent is selected from the group consisting of vitamin B12, thyme dry extract, ivy dry extract, curcuma powder, phenylephrine hydrochloride, dextromethorphan hydrobromide, loratadin, cetirizine, omeprazole, and melatonin.
18. The composition according to any one of items 1 to 17, wherein the particles are prepared by a liquid coating process.
19. The composition according to any one of items 1 to 18, wherein the active ingredient composition is provided in the form of a suspension or a solution of the active ingredient in a liquid diluent.
20. The composition according to item 19, wherein the liquid diluent is water.
21. The composition according to items 19 or 20, wherein the liquid diluent is substantially free of organic solvents.
22. The composition according to any one of items 1 to 21, wherein the particles are substantially free of organic solvents or residues thereof.
23. The composition according to any one of items 1 to 22, wherein the active ingredient composition is applied to the inert starter cores in a fluidized bed coater.
24. The composition according to any one of items 1 to 23, wherein the active ingredient composition comprises an excipient selected from the group of binders, anti-foaming agents, stabilizers, buffers, emulsifiers, solubilizers and anti-flocculation agents.
25. The composition according to any one of items 1 to 24, wherein the active ingredient content of the particles does not exceed 50 wt.-% based on the weight of the active ingredient coated particle.
26. The composition according to any one of items 1 to 25, wherein the active ingredient coated particles comprise at least one further coat of a coating composition, optionally a coat comprising, or consisting of, one or more polymers, waxes and/or lipids applied from aqueous, organic or hot-melt coating compositions.
27. The composition according to item 26, wherein the coated composition exhibits modified release properties, such as taste-masking, enteric release properties and/or sustained release properties.
28. The composition according to any one of items 1 to 27, wherein a single dose unit of the plurality of particles is provided in the form of a tablet compressed therefrom; or a hard-gelatine capsule, stick-pack, sachet, or vial filled with them.
29. A process for the preparation of a dry, flowable composition provided in the form of a plurality particles, wherein the particles each comprise, or consist of, an inert starter core coated with at least one coat of an active ingredient composition, the process comprising the steps of:
   (a) Providing inert starter cores,
   (b) Providing a liquid active ingredient composition,
   (c) Spraying the liquid active ingredient composition onto the inert starter cores, and
   (d) Drying the active ingredient coated particles obtained in step (c),
   characterized in that the active ingredient composition is substantially free of anti-tacking agents, and the inert starter core comprises, or consists of, a sugar alcohol, wherein the sugar alcohol is mannitol provided in a direct compression quality.
30. The process according to item 29, wherein the anti-tacking agent is talc.
31. The process according to item 29 or 30, wherein the inert starter core essentially consists of the mannitol.
32. The process according to any one of items 29 to 31, wherein a plurality of the inert starter cores exhibits a poured density in the range of 0.40-0.80 g/mL, preferably from 0.55-0.80 g/mL, and further preferably from 0.60-0.80 g/mL.
33. The process according to any one of items 29 to 32, wherein a plurality of the inert starter cores exhibits a tapped density in the range of 0.5-1.00 g/mL, preferably 0.60-1.00 g/mL, and further preferably from 0.65-1.00 g/mL.
34. The process according to any one of items 29 to 33, wherein the inert starter core is prepared by an extrusion process, or involving an extrusion process.
35. The process according to any one of items 29 to 34, wherein the inert starter core is prepared by a granulation process, or involving granulation process.
36. The process according to any one of items 29 to 35, wherein the inert starter core is not prepared by a powder coating process.
37. The process according to any one of items 29 to 36, wherein the inert starter core is not prepared by a spray-drying process, or not solely by a spray-drying process.
38. The process according to any one of items 29 to 37, wherein the inert starter core is substantially free of talc.
39. The process according to any one of items 29 to 38, wherein a plurality of the inert starter cores exhibits a median particle size (D50) in the range of about 50-800 μm, or in the range of about 100-500 μm, or in the range of about 150-350 μm, as determined by dynamic image analysis, e.g., using using a Camsizer® XT device (Retsch Technology GmbH, Haan, Germany).
40. The process according to any one of items 29 to 39, wherein the at least one coat of an active ingredient composition is applied directly onto the inert starter cores, i.e., without any intermediate coats.
41. The process according to any one of items 29 to 40, wherein, when measured in water at room temperature (20±5° C.), the active ingredient is considered practically insoluble, very slightly soluble, slightly soluble, sparingly soluble, soluble, or freely soluble as per Ph.Eur. ed.10 definitions; preferably, practically insoluble, very slightly soluble, slightly soluble, sparingly soluble, or soluble.
42. The process according to any one of items 29 to 41, wherein, when measured in water at room temperature (20±5° C.), the active ingredient exhibits an aqueous solubility in the range of 0.01-1000 mg/mL; preferably 0.01-100 mg/mL.
43. The process according to any one of items 29 to 42, wherein the active ingredient is selected from the group consisting of vitamins, plant extracts, cough suppressants, antidiarrhoeals, sleep-inducing agents, antihistaminics, and proton-pump inhibitors.
44. The process according to any one of items 29 to 43, wherein the active ingredient is selected from the group consisting of vitamin B12, thyme dry extract, ivy dry extract, curcuma powder, phenylephrine hydrochloride, dextromethorphan hydrobromide, loratadin, cetirizine, omeprazole, and melatonin.
45. The process according to any one of items 29 to 44, wherein the active ingredient composition is provided in the form of a suspension or a solution of the active ingredient in a liquid diluent.
46. The process according to item 45, wherein the liquid diluent is water.
47. The process according to item 45 or 46, wherein the liquid diluent is substantially free of organic solvents.
48. The process according to any one of items 29 to 47, wherein the particles are substantially free of organic solvents or residues thereof.
49. The process according to any one of items 29 to 48, wherein in step (c) the active ingredient composition is sprayed onto the inert starter cores using a fluidized bed coater.
50. The process according to items 49, wherein the fluidized bed coater is a bottom-spray coater.
51. The process according to item 29 to 50, wherein the spraying pressure in step (c) is in the range of from 1.0 bar to 3.0 bar, preferably 1.4 to 2.0 bar, such as 1.6 bar, 1.7 bar or 1.8 bar.
52. The process according to item 29 to 51, wherein upon using mannitol in direct compression quality as the inert starter cores, the active ingredient coating can be applied by spraying at a maximum spray-rate in g/min per core weight which is at least 1.2× higher, or at least 1.5× higher, or at least 2.0× higher, compared to the same active ingredient coating using the same amount of sucrose-based starter cores instead of the mannitol.
53. The process according to any one of items 29 to 52, wherein the active ingredient composition comprises an excipient selected from the group of binders, anti-foaming agents, stabilizers, buffers, emulsifiers, solubilizers and anti-flocculation agents.
54. The process according to any one of items 29 to 53, wherein the active ingredient content of the particles does not exceed 50 wt.-% based on the weight of the active ingredient coated particle.
55. The process according to any one of items 29 to 54, wherein the active ingredient coated particles comprise at least one further coat of a coating composition, optionally a coat comprising, or consisting of, one or more polymers, waxes and/or lipids applied from aqueous, organic or hot-melt coating compositions.
56. The process according to item 55, wherein the coated composition exhibits modified release properties, such as taste-masking, enteric release properties and/or sustained release properties.
57. The process according to any one of items 29 to 56, wherein the composition is a pharmaceutical or nutraceutical composition.
58. The process according to any one of items 29 to 57, wherein a single dose unit of the composition is provided in the form of a tablet compressed therefrom; or a hard-gelatine capsule, stick-pack, sachet, or vial filled with them.

The following examples serve to illustrate the invention, however should not to be understood as restricting the scope of the invention.

### EXAMPLES

### Example 1A—Coating Vitamin B12 Solution onto Inert Starter Cores

An aqueous solution of vitamin B12 (1 wt.-% based on the weight of the solvent water; crystalline vitamin B12 by DSM Nutritional Products Ltd, Sisseln, Switzerland; solubility of Vit. B12 in water at room temperature: 12.5 mg/mL) was spray-coated without the addition of talc or binder onto 349.3 g of either sucrose starter cores (sugar spheres 45-60 Vivapharm® sugar spheres/JRS Pharma GmbH & Co. KG, Germany; D50 ˜250-300 μm) or mannitol starter cores (Pearlitol® 300DC by Roquette; D50 ˜280-350 μm) using a Ventilus® 2.5 lab-scale, bottom-spray fluidized bed coater equipped with its 1 L product container (Romaco Innojet, Germany). Both starter core materials were initially sieved through an 800 μm sieve prior to applying the active ingredient coat. The spraying pressure was in the range of 0.9-1.1 bar, the air inlet temperature about 70° C., and the air inlet volume in the range of 28-33 m³/h. The aqueous solution was sprayed via a nozzle, using a peristaltic pump to transport the solution, at a spray rate of about 4-5 g/min. The final batch weight of the active ingredient coated particles was approx. 350 g, and the final active ingredient concentration was about 0.2 wt.-% based on the weight of the active ingredient coated particle.

The inventors found that the process worked smoothly and without agglomeration when using the mannitol starter core material, with yields of the ‘good product’ (unagglomerated and passing an 800 μm sieve) in the range of about 97 wt.-% and less than 1 wt-% of the ‘poor product’ (agglomerated particles not passing an 800 μm sieve).

In contrast, the same spray rate of 5 g/min did not work well at all for the sucrose starter cores; instead it led to excessive agglomeration of the starter cores, requiring interruption of the process. Even when reducing the spray rate to only 2.5 g/min in the next coating process (i.e., half the rate used with mannitol starter cores), undesirable agglomerates still formed, thereby significantly reducing the yield with only about 86 wt.-% of ‘good product’ (unagglomerated; passing an 800 μm sieve) and about 13 wt-% of the ‘poor product’ (agglomerated; not passing 800 μm).

This shows that by using mannitol cores in direct compression quality, the active ingredient coating can be applied by spraying at a maximum spray-rate in g/min per core weight which is at least 1.2× higher, or at least 1.5× higher, or at least 2.0× higher, compared to the same active ingredient coating using the same amount of sucrose-based starter cores instead of the mannitol ones.

The issue for sucrose starter cores could only be solved by adding 10 wt.-% talc (based on the weight of the water used to prepare the aqueous active ingredient solution) to the aqueous active ingredient solution; only upon addition of talc, ‘good product’ yields of about 97 wt.-%, similar to the mannitol starter core material, could be achieved.

### Example 1B—Coating Vitamin B12 Solution onto Inert Starter Cores

Similar to example 1A, an aqueous solution of vitamin B12 (1 wt.-% based on the weight of the solvent water; crystalline vitamin B12 by DSM Nutritional Products Ltd, Sisseln, Switzerland), dissolved at about 30-40° C. (e.g., 38° C.), was spray-coated without the addition of talc or binder onto 67,864 kg mannitol starter cores (Pearlitol® 300DC by Roquette; D50 about 280-350 μm) using a Ventilus® V100 industry-scale, bottom-spray fluidized bed coater equipped with its 150 L product container (Romaco Innojet, Germany). The spraying pressure was in the range of 1.5-1.8 bar, the air inlet temperature in the range of about 65-75° C. (e.g., 70° C. or 74° C.), the air inlet volume was about 750 m³/h. The aqueous solution was sprayed via a nozzle at a spraying-air temperature of about 20° C., using a spray rate in the range of about 200-400 g/min, mainly at 300 g/min. The product temperature should stay within a range of about 20-40° C.

The process took about 1 hour in total, including a drying step of max. 30 min. after the B12-solution has been sprayed completely to arrive at a residual moisture in the exhaust air of <20%. The final batch weight of the active ingredient coated particles was approx. 68 kg (67,864 kg mannitol plus 0.136 kg B12), and the final active ingredient concentration was about 0.2 wt.-% based on the weight of the active ingredient coated particle.

The inventors found that the process worked smoothly and without agglomeration when using the mannitol starter core material, with yields of the ‘good product’ (unagglomerated and passing an 800 μm sieve) in the range of about 94-96 wt.-%.

Subsequently, the vitamin B12 coated mannitol cores are removed from the product container and stored until further use, or coated in a next step with a functional coating, such as a hot-melt coating.

### Example 2—Coating Thyme Extract Solution onto Inert Starter Cores

An aqueous suspension of a thyme extract (32 wt.-% based on the weight of the solvent water; Extr. Thymi e Herb. Spir. Sicc. 80% native by Finzelberg GmbH & Co. KG, Germany; very slightly soluble in water at room temperature) was spray-coated without the addition of talc or binders onto 218.75 g of pre-sieved (800 μm) mannitol starter cores (Pearlitol® 300DC by Roquette; D50 ˜280-350 μm) using a Ventilus® 2.5 lab-scale, bottom-spray fluidized bed coater (Romaco Innojet, Germany), equipped with its 1 L product container. The spraying pressure was in the range of 1.4-1.6 bar, the air inlet temperature about 70° C., and the air inlet volume in the range of 35-45 m³/h. The aqueous suspension was sprayed via a nozzle, using a peristaltic pump to transport the solution, at a spray rate of about 4-4.5 g/min. The process worked smoothly and without agglomeration when using the mannitol starter core material without requiring talcum additions. The final batch weight of the active ingredient coated particles was approx. 350 g, and the final active ingredient concentration was about 37.5 wt.-% based on the weight of the active ingredient coated particle.

### Example 3—Coating Phenylephrine HCl Solution onto Inert Starter Cores

An aqueous solution of phenylephrine HCl (30 wt.-% based on the weight of the solvent water; phenylephrine HCl by Syn-Tech Chem. & Pharm. Co., LTD., Taiwan; solubility of phenylephrine HCl in water at room temperature: ≥100 mg/mL) was spray-coated without the addition of talc or binder onto 263.4 g of either sucrose starter cores (sugar spheres 45-60 Vivapharm® sugar spheres/JRS Pharma Gmbh & Co. KG, Germany; 250-355 μm) or mannitol starter cores (Pearlitol® 300DC by Roquette; D50 ˜280-350 μm) using a Ventilus® 2.5 lab-scale, bottom-spray fluidized bed coater, equipped with its 1 L product container (Romaco Innojet, Germany). Both starter core materials were initially sieved through an 800 μm sieve prior to applying the active ingredient coat. The spraying pressure was about 0.9 bar, the air inlet temperature about 70° C., and the air inlet volume in the range of 30-40 m³/h. The aqueous solution was sprayed via a nozzle, using a peristaltic pump to transport the solution, at a spray rate of about 4 g/min. The final batch weight of the active ingredient coated particles was approx. 300 g, and the final active ingredient concentration was about 12.2 wt.-% based on the weight of the active ingredient coated particle.

Same as with the Vitamin B12 in Examples 1A and 1B, the inventors found that the process worked smoothly and without agglomeration when using the mannitol starter core material, with yields of the ‘good product’ (unagglomerated and passing an 800 μm sieve) in the range of about 95 wt.-%.

In contrast, the same spray rate of 4 g/min did not work well at all for the sucrose starter cores; instead it led to excessive agglomeration of the starter cores, requiring interruption of the coating process to allow for further drying; yet even when reducing the spray rate to only 2 g/min upon continuing the coating process (i.e., half the rate used with mannitol starter cores), undesirable agglomerates still formed, thereby significantly reducing the yield with as little as 37 wt.-% of ‘good product’ (unagglomerated; passing an 800 μm sieve) and about 62 wt-% of the ‘poor product’ (agglomerated; not passing 800 μm).

This shows once more that by using mannitol cores in direct compression quality, the active ingredient coating can be applied by spraying at a maximum spray-rate in g/min per core weight which is at least 1.2× higher, or at least 1.5× higher, or at least 2.0× higher, compared to the same active ingredient coating using the same amount of sucrose-based starter cores instead of the mannitol ones.

Even adding 10 wt.-% talc (based on the weight of the water used to prepare the aqueous active ingredient solution) to the aqueous active ingredient solution could improve, yet not sufficiently resolve, the issue with the sucrose starter cores, with the process still showing rather significant agglomeration tendencies and resulting in yields of only about 53 wt.-% ‘good product’ and about 46 wt.-% ‘poor product’.

### Example 4—Coating Melatonin Suspension onto Inert Starter Cores

An aqueous suspension of melatonin (5 wt.-% based on the weight of the solvent water; melatonin by Gonmisol Fine Ingredients, Spain; solubility of melatonin in water at room temperature: 2 mg/mL) was spray-coated without the addition of talc onto 297 g of either sucrose starter cores (sugar spheres 45-60 Vivapharm® sugar spheres/JRS Pharma GmbH &Co. KG, Germany; 250-355 μm) or mannitol starter cores (Pearlitol® 300DC by Roquette; D50 ˜280-350 μm) using a Ventilus® 2.5 lab-scale, bottom-spray fluidized bed coater, equipped with its 1 L product container (Romaco Innojet, Germany). Both starter core materials were initially sieved through an 800 μm sieve prior to applying the active ingredient coat. To improve wettability of melatonin and to prevent excessive foaming of the suspension upon stirring as well as nozzle clogging caused by flocculation of the suspension over time, the suspensions were supplemented with either 0.1 wt.-% Polysorbate 20 or 1 wt.-% hydroxypropyl methylcellulose (HPMC), both based on the weight of the water used to prepare the aqueous active ingredient suspension. The spraying pressure was in the range of about 0.9-1.1 bar, the air inlet temperature about 70° C., and the air inlet volume in the range of 30-35 m³/h. The aqueous suspension was sprayed via a nozzle, using a peristaltic pump to transport the suspension, at a spray rate of about 4 g/min. The final batch weight of the active ingredient coated particles was approx. 300 g, and the final active ingredient concentration was about 1 wt.-% based on the weight of the active ingredient coated particle.

Similar to the findings with Vitamin B12 in Examples 1A and 1B and phenylephrine HCl in Example 3, the inventors found that the process worked smoothly and without agglomeration when using the mannitol starter core material, with yields of the ‘good product’ (unagglomerated and passing an 800 μm sieve) in the range of about 95 wt.-% for both the polysorbate- and the HPMC-supplemented suspensions.

For the sucrose starter cores, the coating process appeared to work well, too, providing even seemingly higher ‘good product’ (unagglomerated; passing an 800 μm sieve) yield of about 98 wt-% and less than 1 wt.-% of the ‘poor product’ (agglomerated; not passing 800 μm) for both the polysorbate- and the HPMC-supplemented suspensions. Yet, upon visual inspection of the sieved ‘good’ fraczion, still a far higher amount of ‘doubles’ and ‘triplets’ was found in the sucrose-based particles (i.e., smaller agglomerates of only two or three particles, respectively) compared to the mannitol based particles. This finding is surprising insofar as the sucrose starter cores are actually meant and marketed for drug/active ingredient coating processes and are deemed far more suited due to their spherical, or at least spheroidal, shape and smooth surfaces.

### Example 5—Particle Size Distribution (PSD) Data

5A) The particle size distribution of six batches of commercially available Pearlitol® 300 DC—one of the preferred mannitol grades in direct compression quality—was determined via dynamic imaging techniques using a Camsizer® XT device (Retsch Technology GmbH, Haan, Germany), equipped with X-Jet plug-in cartridge, using the standard settings of the device, such as according to ISO 13322-2.

Accordingly, the samples were air-dispersed to a density of about 0.3% (w/V) using a dispersion pressure of about 80 kPa and a gap width of 4.0 mm. Upon passing two bright, pulsed LED light sources, the images of the dispersed particles (more specifically of their shadows) were recorded by two digital cameras, analysed for size and shape and subsequently further evaluated with regard to various parameters, including, inter alia, the particle size, more specifically the width of the particle (i.e., shortest chord of the measured set of maximum chords of a particle), fractions of particles within a selected particle size range, D10, D50 and D90, weighted arithmetic mean particle size (mean) and its standard deviation (SD), and aspect ratios (length/width ratio) indicative of sphericity. The results for example 5A are outlined in Table 1 below.

5B) The same test was performed for other mannitol grades in direct compression quality (namely, Pearlitol® 200 GT and Mannogem® XL Ruby); the results of which are are outlined in Table 2 below.

TABLE 1
PSD-data for Pearlitol ® 300 DC (batch to batch variability)

| | batch 1 | batch 2 | batch 3 | batch 4 | batch 5 | batch 6 |
|---|---|---|---|---|---|---|
| D10 | 149.0 | 89.8 | 180.8 | 186.1 | 164.2 | 234.0 |
| D50 | 300.2 | 286.5 | 325.6 | 294.0 | 288.2 | 354.1 |
| D90 | 434.6 | 421.6 | 447.9 | 406.9 | 414.0 | 463.7 |
| mean | 296.5 | 277.0 | 316.7 | 292.5 | 287.6 | 349.6 |
| SD of mean | 109.6 | 117.4 | 109.0 | 92.0 | 99.5 | 91.9 |
| sphericity¹ | 1.37 | 1.37 | 1.35 | 1.32 | 1.33 | 1.33 |
| span² | 0.95 | 1.16 | 0.82 | 0.75 | 0.87 | 0.65 |
| D10/D90 | 0.34 | 0.21 | 0.40 | 0.46 | 0.40 | 0.50 |

¹sphericity = length/width ratio
²span = (D90 − D10)/D50

TABLE 2
PSD-data for Pearlitol ® 200 GT and Mannogem ® XL Ruby

| | 200 GT | XL Ruby |
|---|---|---|
| | (n = 1) | (n = 3) |
| D10 | 77.3 | 104.5 |
| D50 | 126.6 | 231.7 |
| D90 | 209.8 | 407.3 |
| Mean¹ | 139.3 | 242.8 |
| SD of mean | 66.2 | 116.8 |
| sphericity² | 1.23 | 1.41 |
| span³ | 1.05 | 1.31 |
| D10/D90 | 0.37 | 0.26 |

1. A dry, flowable composition provided in the form of a plurality of particles, wherein the particles each comprise, or consist of, an inert starter core coated with at least one coat of an active ingredient composition, characterized in that the active ingredient composition is substantially free of anti-tacking agents, and the inert starter core comprises, or consists of, a sugar alcohol, wherein the sugar alcohol is mannitol provided in a direct compression quality.

2. The composition according to claim 1, wherein the anti-tacking agent is talc.

3. The composition according to claim 1, wherein the inert starter core essentially consists of the mannitol provided in a direct compression quality.

4. The composition according to claim 1, wherein a plurality of the inert starter cores exhibits a poured density in the range of 0.40-0.80 g/mL, 0.55-0.80 g/mL, or 0.60-0.80 g/mL; and/or a tapped density in the range of 0.50-1.00 g/mL, 0.60-1.00 g/mL, or 0.65-1.00 g/mL.

5. The composition according to claim 1, wherein the inert starter core is substantially free of talc.

6. The composition according to claim 1, wherein a plurality of the inert starter cores exhibits a median particle size (D50) in the range of about 50-800 μm, or in the range of about 100-500 μm, or in the range of about 150-350 μm, as determined by dynamic image analysis.

7. The composition according to claim 1, wherein the composition is a pharmaceutical or nutraceutical composition.

8. The composition according to claim 1, wherein the active ingredient is selected from the group consisting of vitamins, plant extracts, sympathomimetics, analgesics, cough suppressants, antidiarrhoeals, sleep-inducing agents, anti-histaminics, and proton-pump inhibitors.

9. The composition according to claim 1, wherein the active ingredient content of the particles does not exceed 50 wt.-% based on the weight of the active ingredient coated particle.

10. The composition according to claim 1, wherein the active ingredient coated particles comprise at least one further coat of a coating composition.

11. The composition according to claim 1, wherein a single dose unit of the plurality of particles is provided in the form of a tablet compressed therefrom; or a hard-gelatine capsule, stick-pack, sachet, or vial filled with them.

12. A process for the preparation of a dry, flowable composition provided in the form of a plurality particles, wherein the particles each comprise, or consist of, an inert starter core coated with at least one coat of an active ingredient composition, the process comprising the steps of:
(a) providing inert starter cores,
(b) providing a liquid active ingredient composition,
(c) spraying the liquid active ingredient composition onto the inert starter cores, and
(d) drying the active ingredient coated particles obtained in step (c),
characterized in that the active ingredient composition is substantially free of anti-tacking agents, and the inert starter core comprises, or consists of, a sugar alcohol, wherein the sugar alcohol is mannitol provided in a direct compression quality.

13. The process according to claim 12, wherein the active ingredient composition is provided in the form of a suspension or a solution of the active ingredient in a liquid diluent.

14. The process according to claim 13, wherein the liquid diluent is water, and wherein the liquid diluent is substantially free of organic solvents.

15. The process according to claim 12, wherein in step (c) the active ingredient composition is sprayed onto the inert starter cores using a fluidized bed coater.

16. The process according to claim 12, wherein the anti-tacking agent is talc.

17. The process according to claim 13, wherein the liquid diluent is water.

18. The process according to claim 15, wherein the fluidized bed coater is a bottom-spray coater.
